(19) United States
(12) Patent Application Publication
(10) Pub. No.: US 2026/0345889 A1
(43) Pub. Date: Nov. 5, 2026

(54) FLOW AUGMENTATION FOR DRAINAGE CATHETERS

(71) Applicant: BOARD OF REGENTS, THE UNIVERSITY OF TEXAS SYSTEM, Austin, TX (US)

(72) Inventor: Ravi MURTHY, Houston, TX (US)

(21) Appl. No.: 19/487,226

(22) PCT Filed: May 24, 2024
(86) PCT No.: PCT/US2024/030921

§ 371 Date: Nov. 24, 2025

Related U.S. Application Data
(60) Provisional application No. 63/503,973, filed on May 24, 2023.

(51) Int. Cl. A61M 1/00

(52) U.S. Cl. CPC A61M 1/84; A61M 2202/0014; A61M 2202/04; A61M 2205/04; A61M 2205/103; A61M 2205/3327; A61M 2205/3331; A61M 2205/3379; A61M 2205/8206; A61M 2205/8281; A61M 2206/16; A61M 2206/20; A61M 2210/1071; A61M 2210/1078

(57) ABSTRACT

Systems and methods for flow augmentation in drainage catheters. In one example, a method of facilitating in vivo liquid flow in a drain passageway having an obstruction includes implanting a drain catheter and an elongated rotatable drive in the drain passageway through the obstruction, with the elongated rotatable drive extending along a lumen of the drain catheter. The elongated rotatable drive is rotated such that liquid in the drain passageway is drawn into the drain catheter upstream of the obstruction and exits the drain catheter downstream of the obstruction.

WO

### CROSS-REFERENCE TO RELATED APPLICATIONS

This application claims priority to and the benefit of the filing date of U.S. provisional patent application Ser. No. 63/503,973 filed May 24, 2023 for “Flow Augmentation for Drainage Catheters,” the entire contents of which are hereby incorporated by this reference.

### RELATED FIELDS

Treatment of tumor-related or other mechanical obstructions to the normal flow of bile, urine, or other fluids generated by the body.

### BACKGROUND

In some instances, tumor-related obstructions are bypassed by placing a perforated hollow catheter through the tumor itself. In principle, fluid can pass from one side of the obstructing tumor to the other side of the tumor by entering the catheter through the perforations on the side generating the fluid and exiting the catheter via perforations on the opposite side of the tumor. This can alleviate obstruction/blockage. However, in many instances, the catheter will become clogged and/or leak at the insertion site, leading to organ malfunction and the development of infection.

### SUMMARY

In one example, an elongated, flexible helical screw is designed for insertion into a catheter and remains in place in the catheter during use. The screw is associated with a motor that turns the screw intermittently or continuously. In some implementations, the screw may be rotated at an interval of one minute on, one minute off (which may beneficially extend battery life for the motor). In use, the motor rotates the screw so that a small volume of fluid is allowed to pass through the catheter along the rotations of the screw. In some implementations, a rate of fluid flow around 1 cc/minute may suffice for clinical applications for this device. In some implementations, a rate of fluid flow around 0.5 cc/minute to 5 cc/minute may suffice for clinical applications for this device.

The rotating screw may be designed so it can be used with various types of commercially available catheters on the market. It may be designed to stay positioned within the catheter during the life of its use and may be disposed of with the catheter when use is complete. It is also envisioned that the motor may be disposable in order to alleviate the need for autoclaving or other forms of sterilization.

In one example a method of facilitating in vivo liquid flow in a drain passageway having an obstruction includes implanting a drain catheter and an elongated rotatable drive in the drain passageway. The drain catheter extends through the obstruction and the elongated rotatable drive extends along a lumen of the drain catheter. The method further includes rotating the elongated rotatable drive such that liquid in the drain passageway is drawn into the drain catheter upstream of the obstruction and exits the drain catheter downstream of the obstruction.

In some implementations the drain passageway comprises an in vivo bile duct or an in vivo ureter.

In some implementations the obstruction is a tumor.

In some implementations the obstruction is a stenosis of the drain passageway.

In some implementations the elongated rotatable drive is implanted into the drain catheter after implantation of the drain catheter.

In some implementations the drain catheter includes at least one lateral opening extending from an exterior of the drain catheter to the lumen.

In some implementations, after implantation of the drain catheter, at least one lateral opening of the drain catheter is located upstream of the obstruction.

In some implementations, after implantation of the elongated rotatable drive, during rotation of the elongated rotatable drive, liquid in the drain passageway enters into the drain catheter through the at least one upstream opening and exits the drain catheter downstream of the obstruction.

In some implementations the elongated rotatable drive remains in the drain catheter as the liquid drains through the drain catheter.

In some implementations the liquid flows unidirectionally through the drain catheter during rotation of the elongated rotatable drive and exits both through a lateral openings downstream of the obstruction and through a distal end of the drain catheter.

In some implementations the elongated rotatable drive includes a helical screw drive portion.

In some implementations, after implantation of the elongated rotatable drive, the screw drive portion extends through the obstruction and past at least one upstream opening and at least one downstream opening of the drain catheter.

In some implementations, after implantation of the elongated rotatable drive, the screw drive portion is located at or upstream of the obstruction.

In some implementations, after implantation of the elongated rotatable drive, the screw drive portion is located proximate the at least one opening of the drain catheter upstream of the obstruction.

In some implementations, the elongated rotatable drive is coaxially inserted in the drain catheter.

In some implementations, prior to implantation of the elongated rotatable drive, an end segment of the elongated rotatable drive is trimmed off to shorten the elongated rotatable drive.

In some implementations the method includes connecting the elongated rotatable drive to a drive motor.

In some implementations the drive motor periodically or continuously rotates the elongated rotatable drive.

In some implementations the drive motor is energized by a battery or a spring (such as a mechanical internal mainspring).

In some implementations the elongated rotatable drive is resiliently bendable.

In some implementations the method includes quantifying a volume of fluid that has flowed through the drain catheter using a flow sensor associated with at least either the drain catheter or the elongated rotatable drive.

In some implementations the method includes monitoring performance of the drain catheter using a feedback sensor.

In another example a system for facilitating in vivo liquid flow in a drain passageway having an obstruction includes an elongated drain catheter. The catheter has a lumen, an exterior, and a series of lateral openings extending from the exterior to the lumen. The system also includes an elongated rotatable drive with a screw drive portion configured for insertion into the lumen of the drain catheter such that the screw drive portion extends past at least one of the lateral openings. The system also includes a drive motor configured to rotate the elongated rotatable drive.

In some implementations the elongated rotatable drive is trimmable.

In some implementations the screw drive portion is resiliently bendable.

### BRIEF DESCRIPTION OF FIGURES

FIGS. 1-3 show an example of a system for facilitating in vivo flow in a drain passageway having an obstruction. FIG. 1 shows an elongated rotatable drive and drive motor of the system. FIG. 2 shows the elongated rotatable drive of FIG. 1 coaxially inserted into an elongated drain catheter. FIG. 3 shows the system in vivo.

FIG. 4 shows an example of a drain passageway with an obstruction.

FIG. 5 shows an example of a system of facilitating in vivo liquid flow in the drain passageway of FIG. 4.

FIGS. 6 and 7 show another example of a system for facilitating in vivo flow in a drain passageway having an obstruction.

FIGS. 8-11 show an example of a screw drive portion of an elongated rotatable drive.

FIG. 12 shows another example of a screw drive portion.

FIG. 13 shows experimental flow rates associated with a single helix screw drive portion.

FIG. 14 shows another example of a screw drive portion.

FIG. 15 shows experimental flow rates associated with a double helix screw drive portion.

FIGS. 16-22 schematically illustrate an example of a flow augmentation method.

FIGS. 23-24 illustrate an experiment demonstrating the effectiveness of a flow augmentation system.

### DETAILED DESCRIPTION

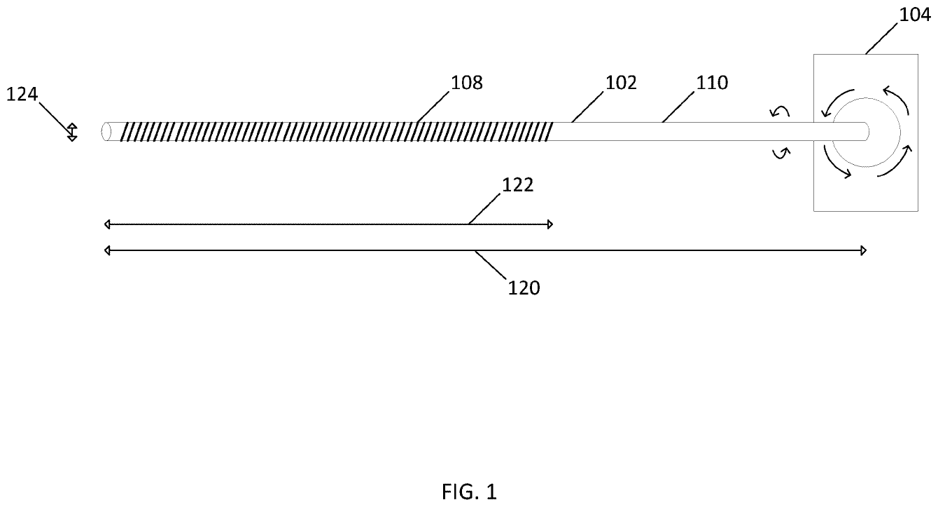

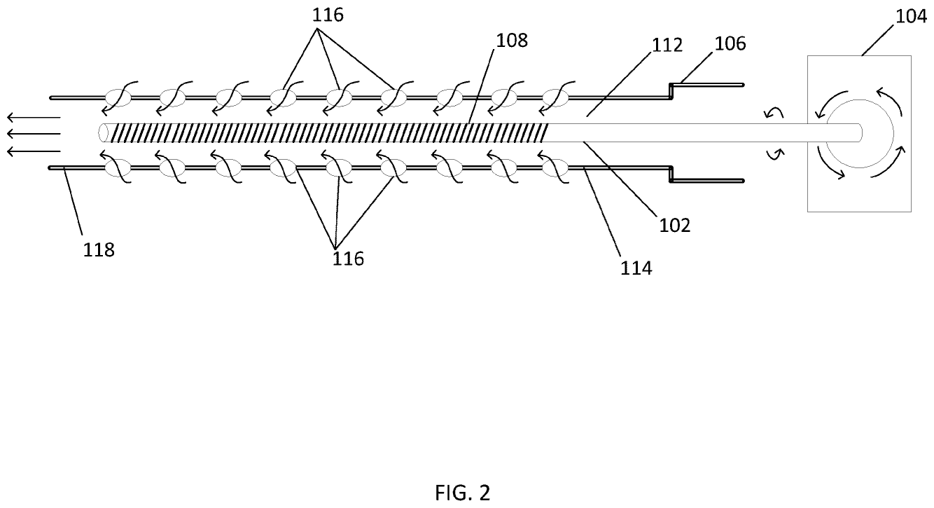

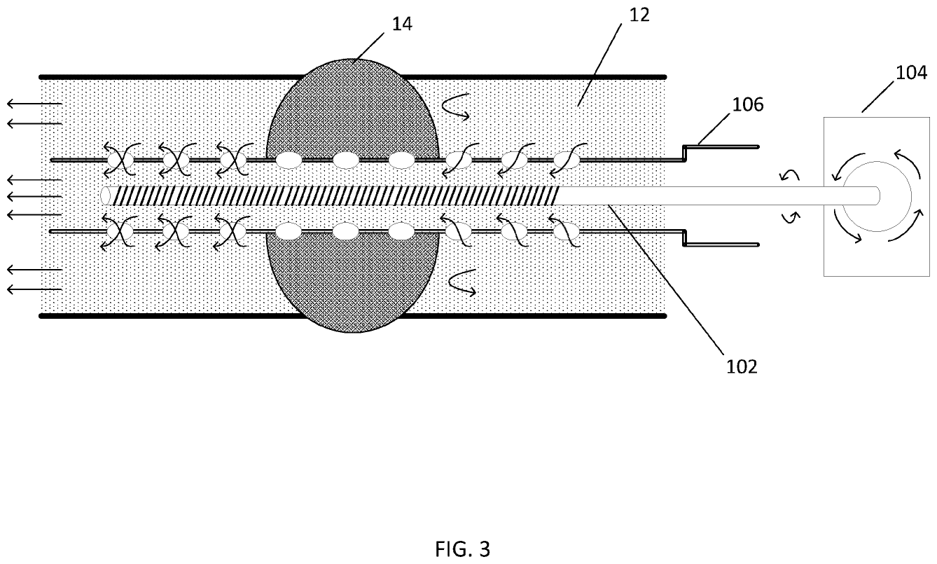

FIGS. 1-3 show an example of a system for facilitating in vivo flow in a drain passageway having an obstruction. FIG. 1 shows a drive motor 104 configured to rotate an elongated rotatable drive 102. FIG. 2 shows the elongated rotatable drive 102 inserted into an elongated drain catheter 106. FIG. 3 shows the elongated rotatable drive 102 and drain catheter 106 implanted in a drain passageway 12 having an obstruction 14.

In FIG. 1, the elongated rotatable drive 102 includes a helical screw drive portion 108 extending partially along the length of the drive 102. In some implementations the screw drive portion 108 may be a helical screw such as an Archimedes screw or otherwise configured to function as an Archimedes screw or another type of helical or other drive that will facilitate driving fluids along the screw drive portion 108 when the elongated rotatable drive 102 is rotated. In some implementations the elongated rotatable drive 102 may be a trimmable, resiliently bendable material such as a plastic or a metallic alloy such as nitinol. An end segment of the elongated rotatable drive 102 may be trimmed off to shorten the length of the drive for the particular circumstances (e.g. for the particular length of drain catheter and/or particular length of drain passageway in which it will be used). Because elongated rotatable drive 102 is resiliently bendable, it can be used with drain catheters and drain passageways that are not straight. In some implementations the overall length (reference 120 in FIG. 1) of the elongated drive 102 may be in the range of 25-30 cm, 20-35 cm, 15-40 cm, or other lengths. In some implementations the length (reference 122) of screw drive portion 108 may be approximately 15 cm, in the range of 10-20 cm, or other lengths. In some implementations the diameter (reference 124) of the elongated drive 102 may be approximately 4-5 Fr, 3-6 Fr, or other diameters.

In still other implementations the screw drive portion may be substantially shorter and positioned towards the end of elongated drive.

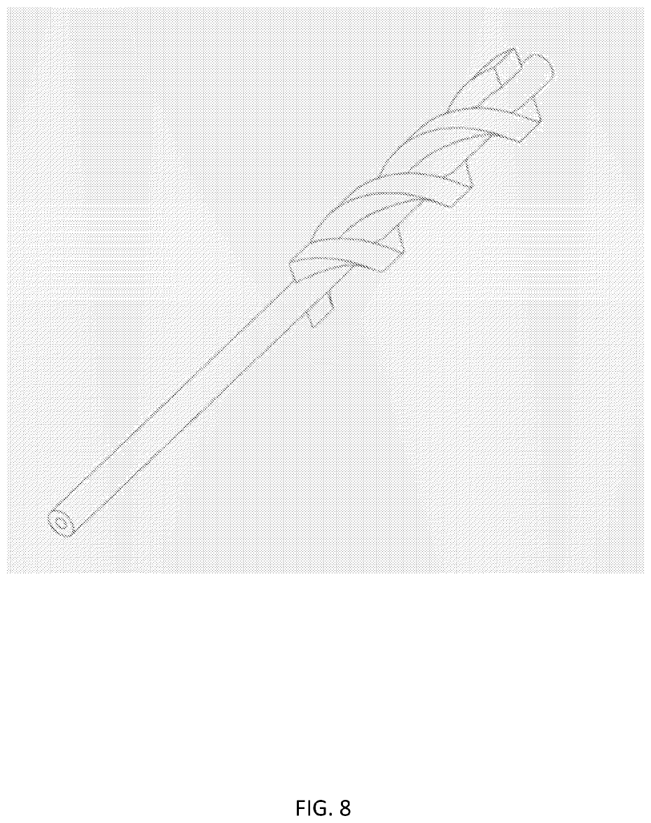

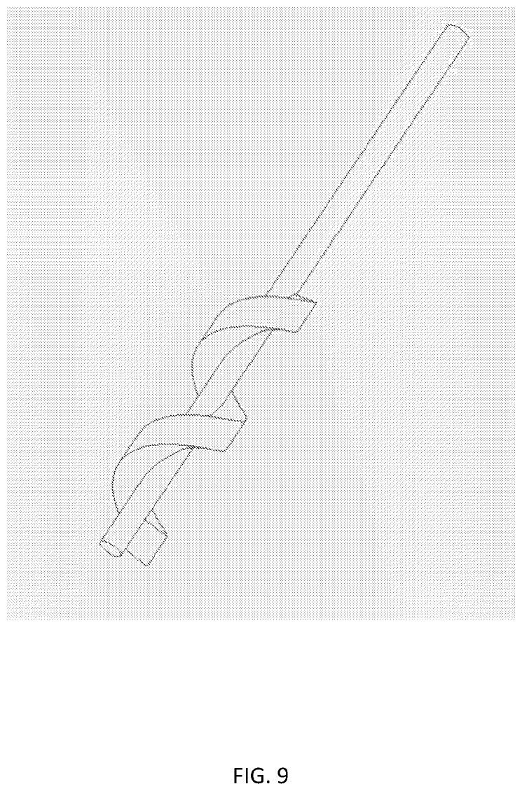

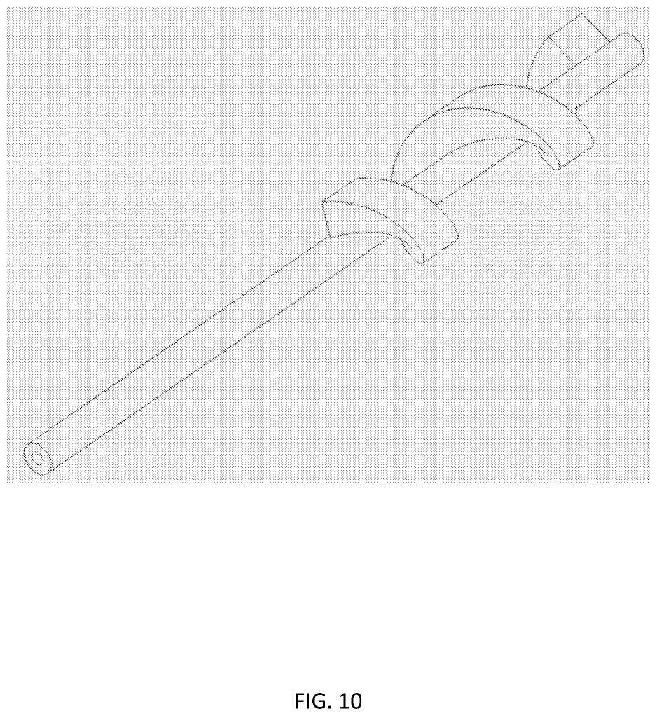

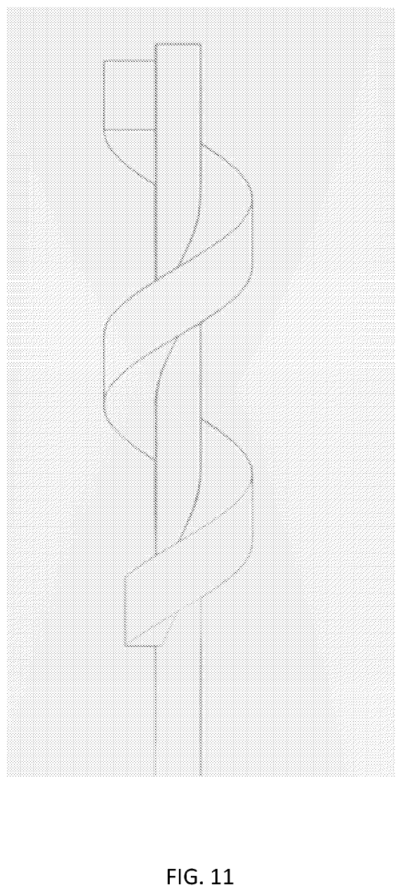

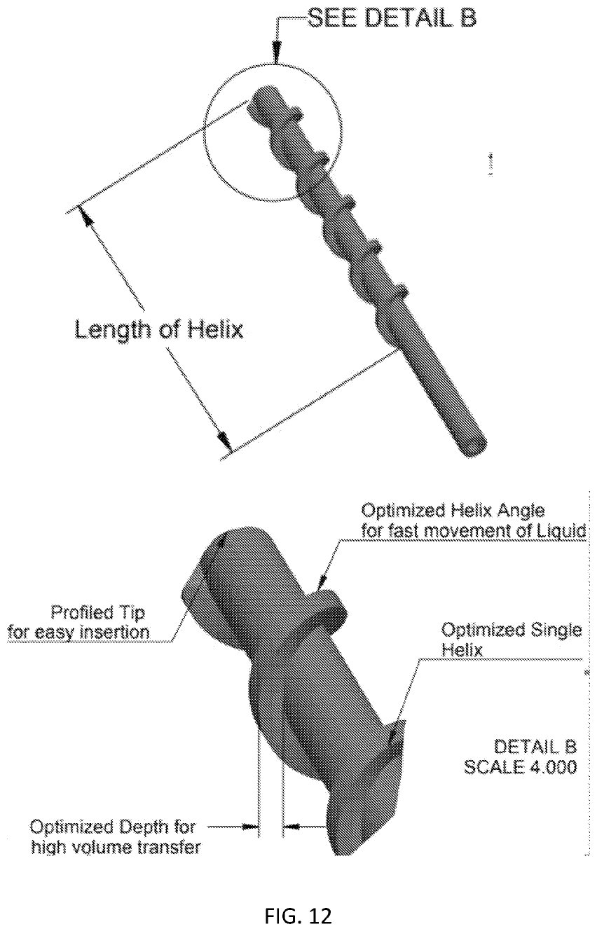

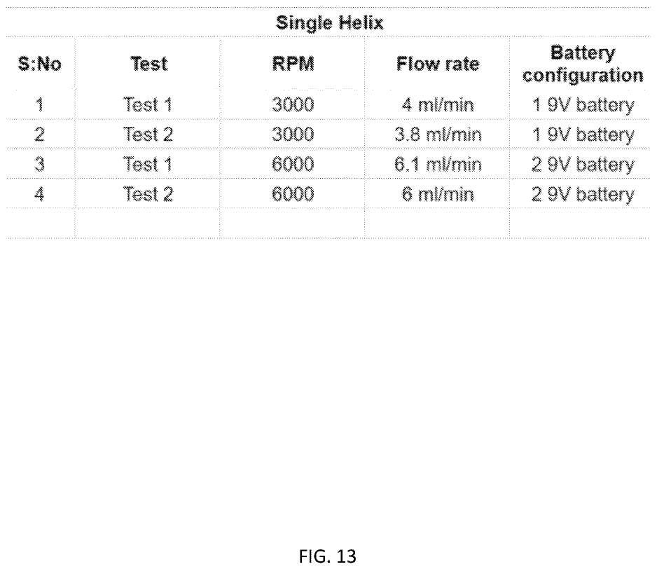

FIGS. 8-11 show an example of a single helix screw drive portion on the end of an elongated drive (with only a portion of the elongated drive shown). FIG. 12 shows another example of a single helix screw drive portion on the end of an elongated drive (with only a portion of the elongate drive shown). As shown in FIG. 12, the tip of the elongated drive may have a rounded or similar profile to ease insertion into the drain catheter. The helical geometry (including helical depth, angle, and length) may all be optimized for volume and speed of fluid movement through the catheter. FIG. 13 shows test data for two different configurations of a single helix system. As shown, the single helix may provide flow rates of between 4 ml/min and 6.1 ml/min depending on the RPM (rotations per minute) and voltage configuration applied to the device. In other implementations single helix devices may be configured to provide flow rates between 2-10 ml/min or at other rates.

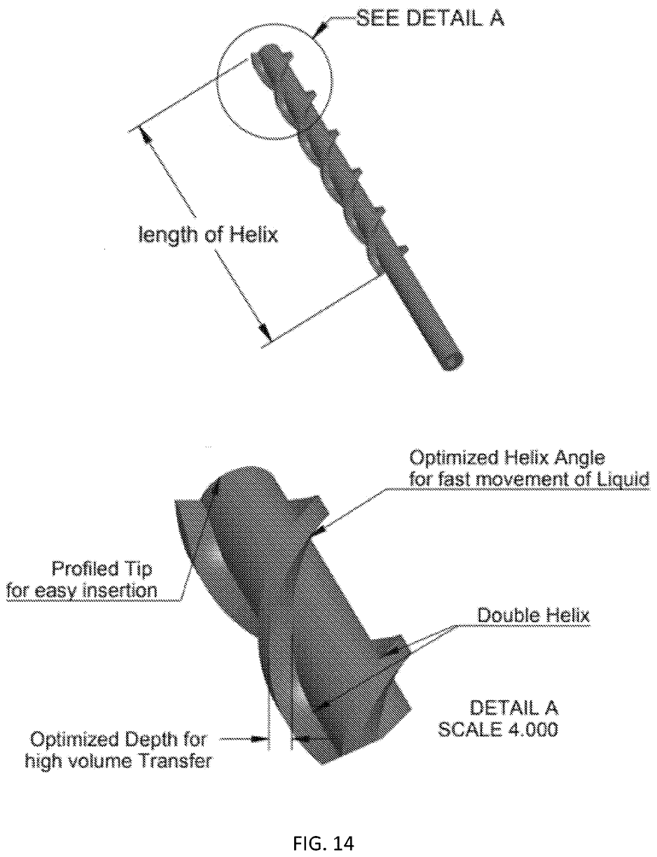

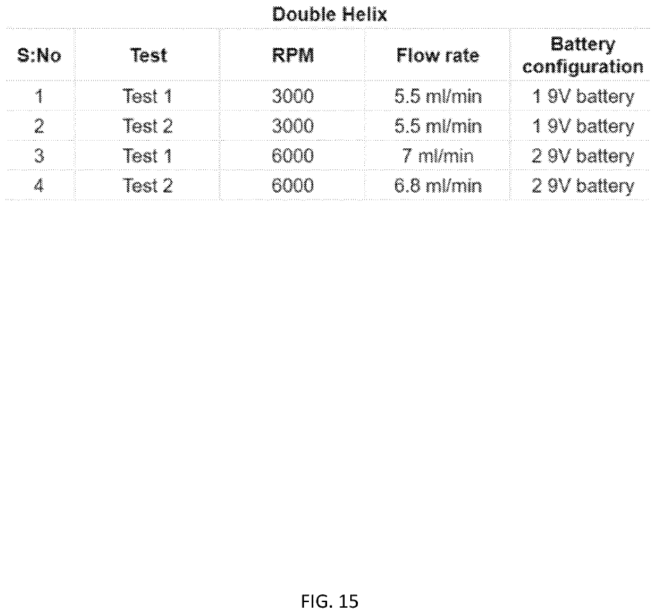

FIG. 14 shows an example of a double helix screw drive (i.e. two separate helixes wound around the elongated drive) with only a portion of the elongated drive shown. As with the single helix drive examples discussed above, the tip of the elongated drive may have a rounded or similar profile to ease insertion into the drain catheter. The helical geometry (including helical depth, angle, and length) may all be optimized for volume and speed of fluid movement through the catheter. FIG. 15 shows test data for two different configurations of a double helix device. As shown, the single helix device may provide flow rates of between 5.5 ml/min and 7 ml/min depending on the RPM and voltage configuration of the device. In other implementations double helix devices may be configured to provide flow rates between 2-10 ml/min or at other rates.

Returning to the example of FIG. 1, the elongated rotatable drive 102 has an unthreaded drive shaft portion 110. The elongated rotatable drive 102 is connected at the end of the drive shaft portion 110 to the drive motor 104. Drive motor 104 may be configured to periodically or continuously rotate the elongated rotatable drive 102. In some implementations the drive motor 104 may be a DC motor energized by a battery (not shown).

In the example of FIG. 2 the elongated rotatable drive 102 has been inserted into the elongated drain catheter 106. The elongated rotatable drive 102 may be inserted into the elongated drain catheter 106 prior to implantation of the catheter 106 into a patient such that the drive 102 and catheter 106 are implanted at the same time. In other implementations the drive 102 may be inserted into the catheter 106 after the catheter 106 has already been implanted into the patient.

The drain catheter 106 in FIG. 2 includes a lumen 112, an exterior 114, and several lateral openings 116 extending from the exterior 114 to the lumen 112. The drain catheter 106 is also open at its distal end 118.

In the example of FIG. 2, when the elongated rotatable drive 102 is coaxially inserted into and extends along the lumen 112 of the drain catheter 106, the screw drive portion 108 extends past all the lateral openings 116. In other implementations, the screw drive portion 108 extends past just some or even just one of the lateral openings 116.

FIG. 3 shows an example of use for the elongated rotatable drive 102, drive motor 104, and elongated drain catheter 106 of FIGS. 1 and 2. In the example of FIG. 3, the catheter 106 and drive 102 have been implanted in a drain passageway 12 through an obstruction 14. After implantation, at least some of the lateral openings and part of screw drive portion of elongated drive 102 are located upstream of the obstruction 14 and at least some of the lateral openings and part of screw drive portion of elongated drive 102 are located downstream of the obstruction 14. Drive motor 104 rotates elongated drive 102 such that liquid in the drain passageway 12 is drawn into the catheter 106 through lateral openings upstream of the obstruction 14 and exits the catheter 106 downstream of the obstruction 14. In other words, liquid flows unidirectionally through the catheter 106 during rotation of the elongated drive 102. In this implementation the liquid exits the catheter 106 both through the lateral openings downstream of obstruction 14 and through the distal end opening.

In this manner, in vivo liquid flow is facilitated in the drain passageway 12 despite obstruction 14. The elongated rotatable drive 102 remains in the drain catheter 106 as the liquid drains through the catheter 106, and the drive motor 104 may be periodically or continuously operated to facilitate maintaining the functionality of the catheter 106.

In some implementations the drain passageway 12 may be an in vivo bile duct or ureter. In some implementations the obstruction 14 may be a tumor or other stenosis of the drain passageway 12.

While not shown in the figures, in some implementations the elongated drive 102 and/or drain catheter 106 may include a flow sensor to facilitate quantification of a volume of fluid that has flowed through the catheter 106. In some implementations the flow sensor or other sensors may be used to monitor performance of the catheter 106 or otherwise provide feedback indicating that the system is or is not functioning properly.

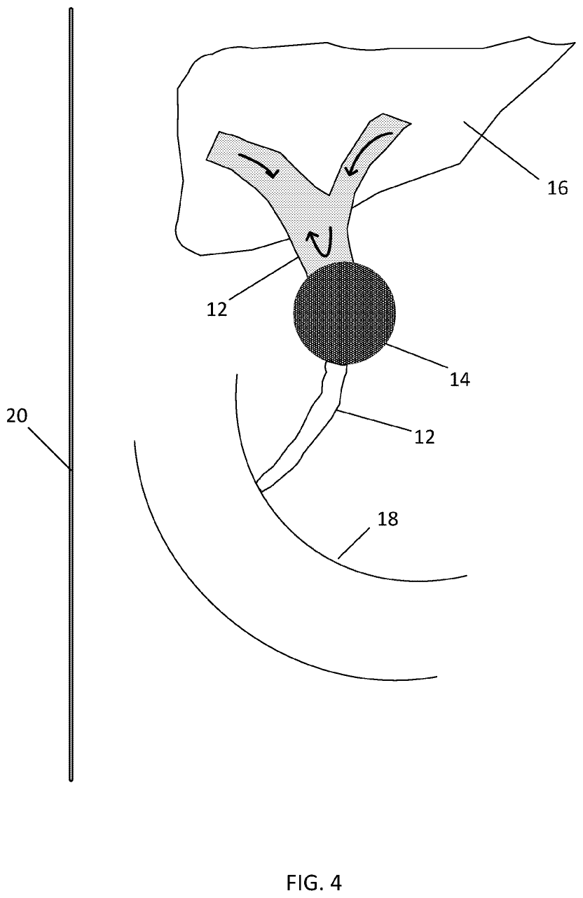

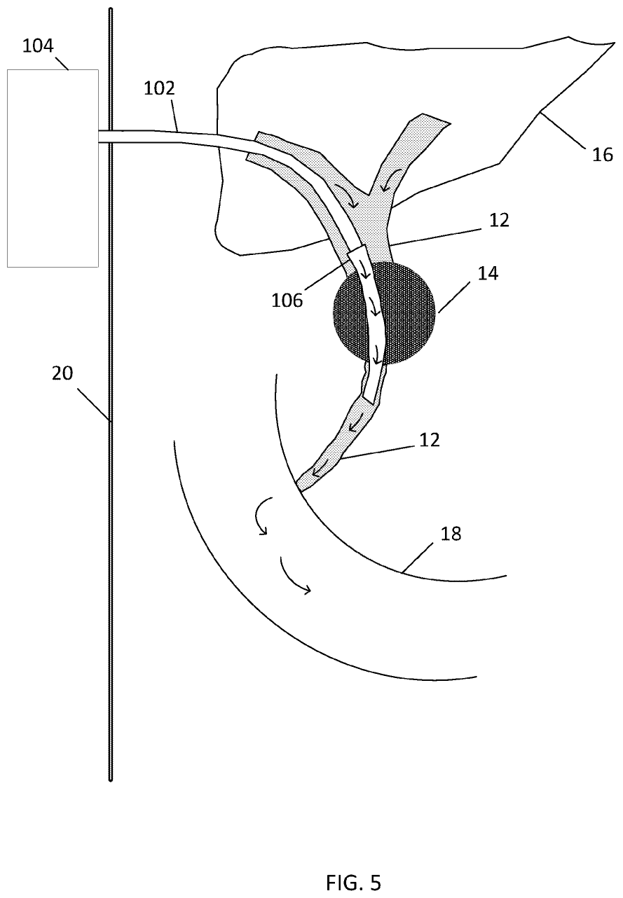

FIGS. 4 and 5 illustrate an example use case for the system of FIGS. 1 and 2. In FIG. 4 the drain passageway 12 is a bile duct extending from the liver 16 to the duodenum 18. A tumor 14 obstructs the drain passageway 12 preventing or at least hampering proper flow of bile from the liver 16 to the duodenum 18.

FIG. 5 shows the system of FIGS. 1 and 2 used to re-establish proper liquid flow. Elongated drain catheter 106 and elongated drive 102 been implanted into the bile duct 12 through tumor 14. Drive motor 104 is located extracorporeally and connects to the proximal end of elongated drive 102 extending through the patient's skin 20.

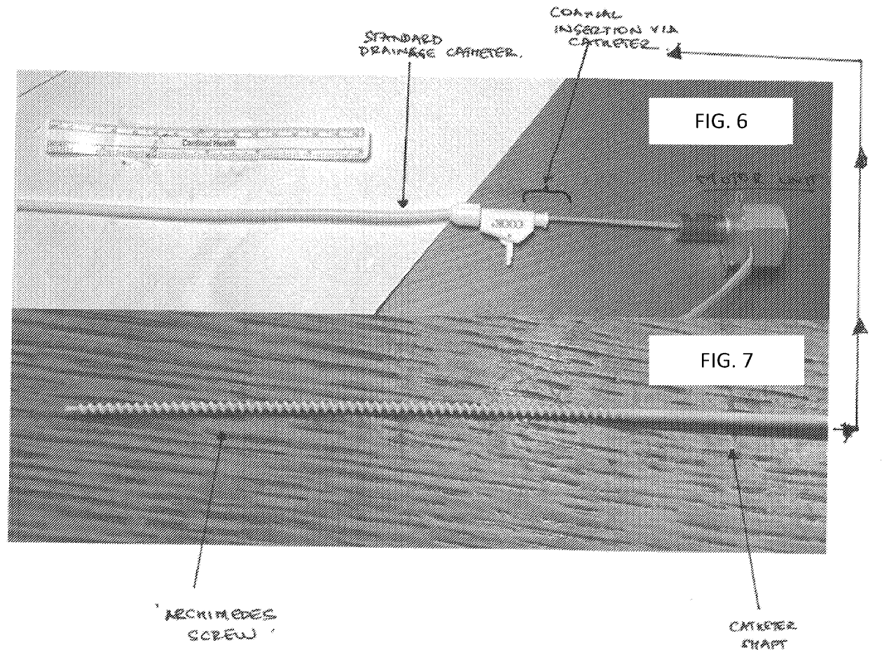

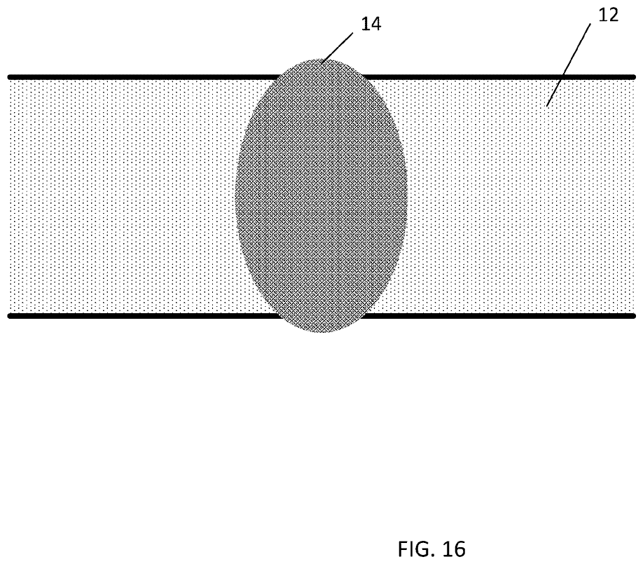

FIGS. 16-21 show another example of a method of facilitating in vivo liquid flow in a drain passageway 12 having an obstruction 14 using an elongate rotatable drive 102 and a drain catheter 106. In this particular example elongate rotatable drive 102 has a relatively short screw drive portion 126 such as the single and double helix screw drive portions illustrated in FIGS. 8-15. In some instances the longer Archimedes type screw shown in FIGS. 1-7 may be susceptible to vapor lock or other limitations in performance, especially in environments where the entire length of the screw drive portion is not uniformly immersed in liquid, as can often be the case in drain passageways. The relatively short screw drive portion 126 in FIGS. 16-21 may be less susceptible to these performance limitations. In some implementations the screw drive portion 126 may be less than 5 cm in length, less than 3 cm in length, or less than 2 cm in length.

In the method of FIGS. 16-21 the drain catheter 106 and elongated rotatable drive 102 are implanted in the drain passageway 12 through the obstruction 14, with the elongated rotatable drive 102 extending along a lumen 112 of the drain catheter 106. Rotation of the elongated rotatable drive 102 facilitates liquid in the drain passageway 12 being drawn into the drain catheter 106 upstream of the obstruction 14 and exiting the drain catheter 106 downstream of the obstruction 14.

FIG. 16 shows a drain passageway 12 with an obstruction 14. In FIG. 16 the obstruction 14 is shown as a structure that physically extends through and completely blocks the drain passageway 12 but those of skill in the art will appreciate that obstruction 14 could be any stenosis or other abnormal narrowing of or blocking of the drain passageway 12 (e.g., such as caused by a tumorous growth in proximity of the drain passageway).

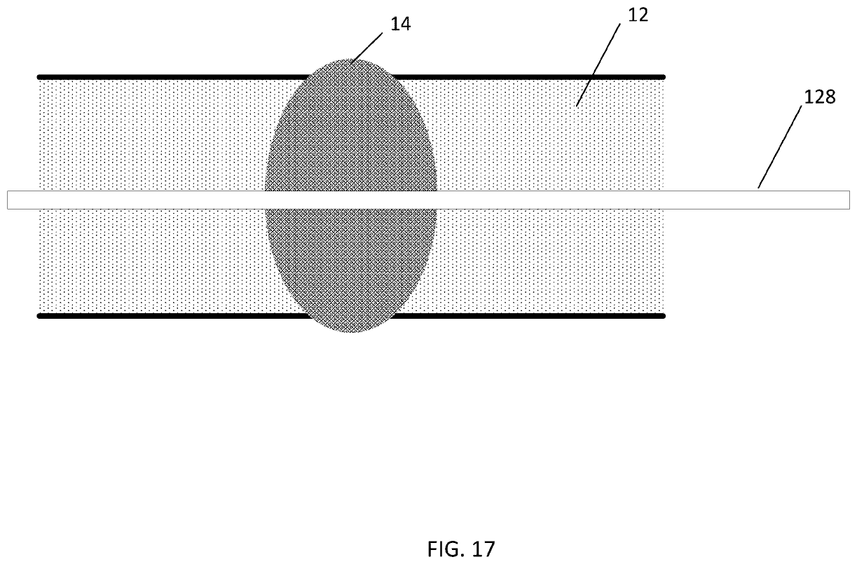

FIG. 17 shows a guidewire 128 inserted into the drain passageway 12 and through obstruction 14.

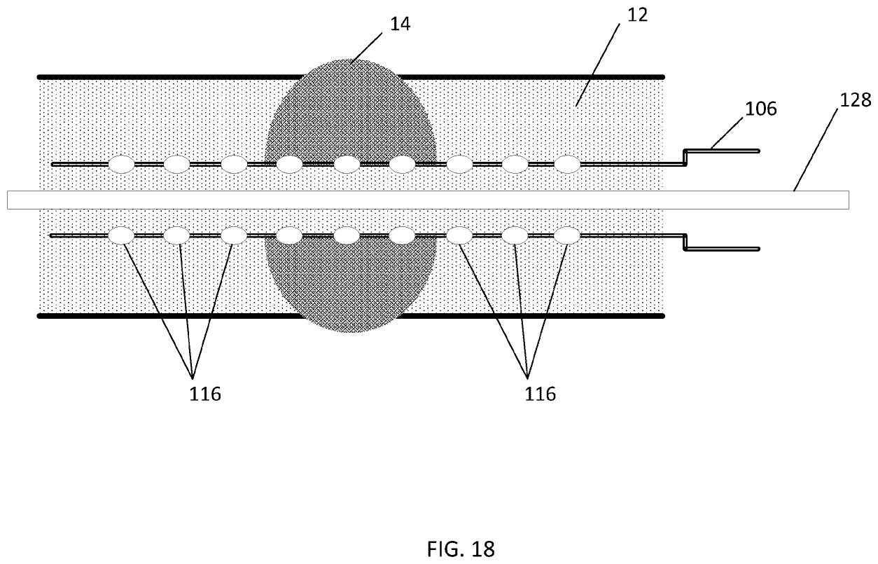

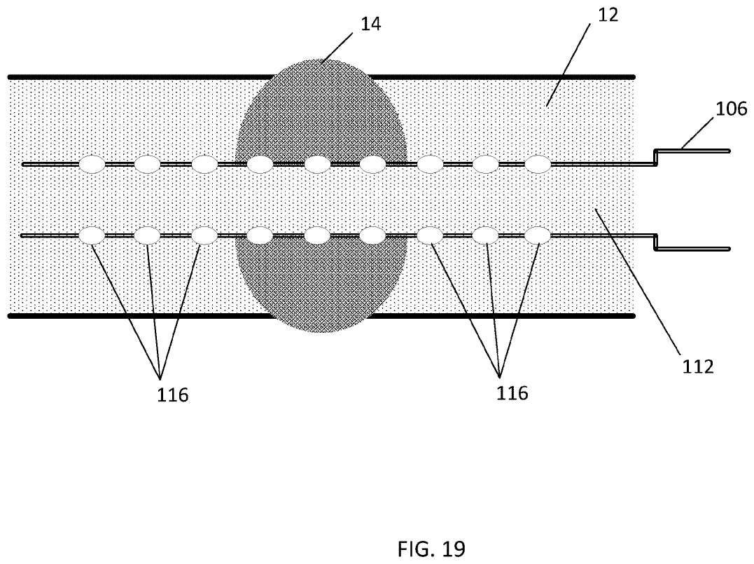

Guidewire 128 may be used to guide implanting the drain catheter 106 in the drain passageway 12 and through the obstruction 14 as shown in FIG. 18. The guidewire 128 may be removed from the drain passageway 12 after implanting the drain catheter 106 as shown in FIG. 19. As shown in FIG. 19, drain catheter 106 is implanted such that one or more lateral openings 116 of the drain catheter 106 are located upstream of the obstruction 14 and one or more lateral openings 116 of the drain catheter 106 are located downstream of the obstruction 14.

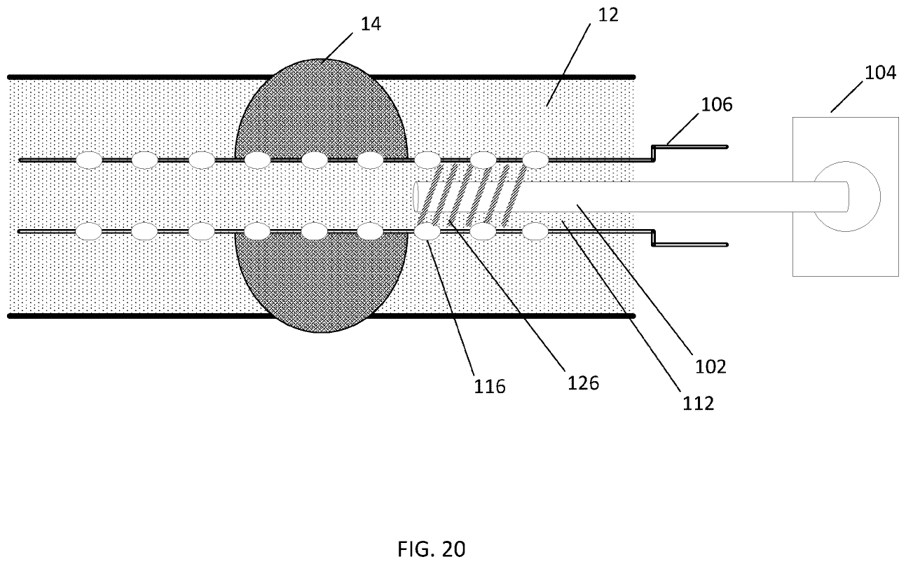

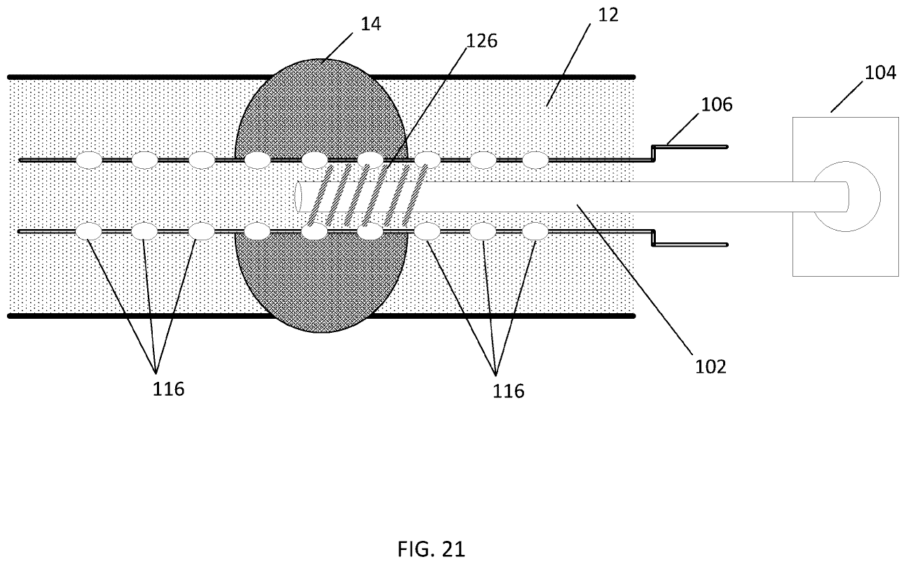

As shown in FIG. 20 the elongated rotatable drive 102 may subsequently be coaxially inserted in the drain catheter 106. As schematically shown in FIG. 20 the elongated rotatable drive 102 is coaxially inserted in the drain catheter 106 such that the screw drive portion 126 is proximate the lateral openings 116 upstream of the obstruction 14. In other implementations, and as schematically shown in FIG. 21, the elongated rotatable drive 102 is coaxially inserted in the drain catheter 106 such that the screw drive portion 126 is proximate the obstruction 14 and between at least some of the lateral openings 116 upstream of the obstruction 14 and some of the lateral openings 116 downstream of the obstruction 14. Intraoperative imaging or other suitable techniques may be used to confirm the desired positioning of the screw drive portion 126 relative to the drain catheter 106, the lateral openings 116 in the drain catheter 106, and/or the obstruction 14.

As shown in FIGS. 20 and 21, the screw drive portions 126 in these examples are significantly shorter than the screw drive portion 108 in the earlier example, such as shown in FIG. 3. In the example of FIG. 3, the screw drive portion 108 spans lateral openings 116 in the drain catheter 106 that are both upstream and downstream of the obstruction 14. In the examples of FIGS. 20 and 21, the screw drive portion 126 does not span lateral openings 116 in the drain catheter 106 both upstream and downstream of the obstruction 14.

As shown in FIGS. 20 and 21, in these examples, the outer diameter of the screw drive portion 126 may be approximately equal to or slightly less than the inner diameter of the drain catheter 106. In some implementations the outer diameter of the screw drive portion 126 may be at least 80% of the inner diameter of the drain catheter 106, at least 90% of the inner diameter of the drain catheter 106, or at least 95% of the inner diameter of the drain catheter 106.

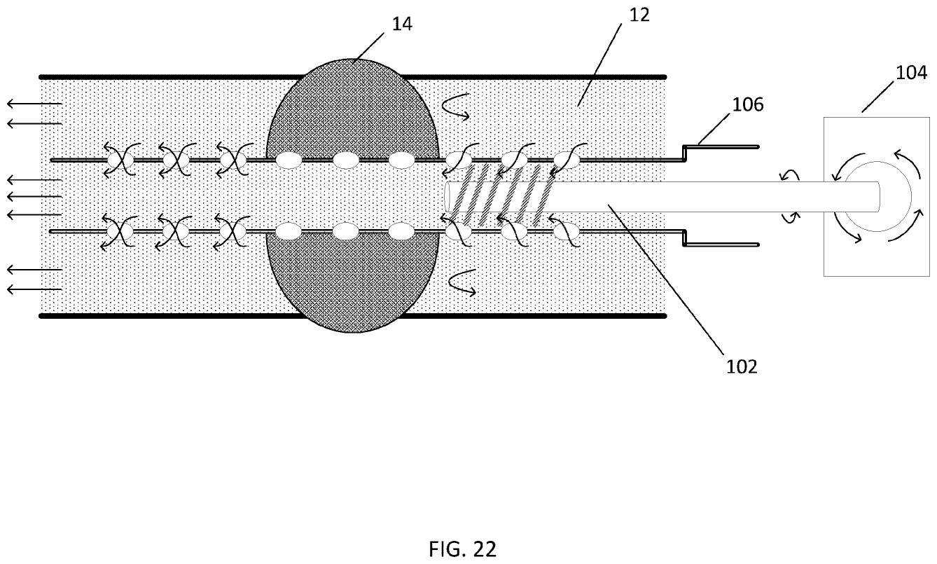

As shown in FIG. 22, after insertion of the elongated rotatable drive 102 into the drain catheter 106, the elongated rotatable drive 106 is rotated such that liquid in the drain passageway 12 is drawn into the drain catheter 106 upstream of the obstruction 14 and exits the drain catheter 106 downstream of the obstruction 14.

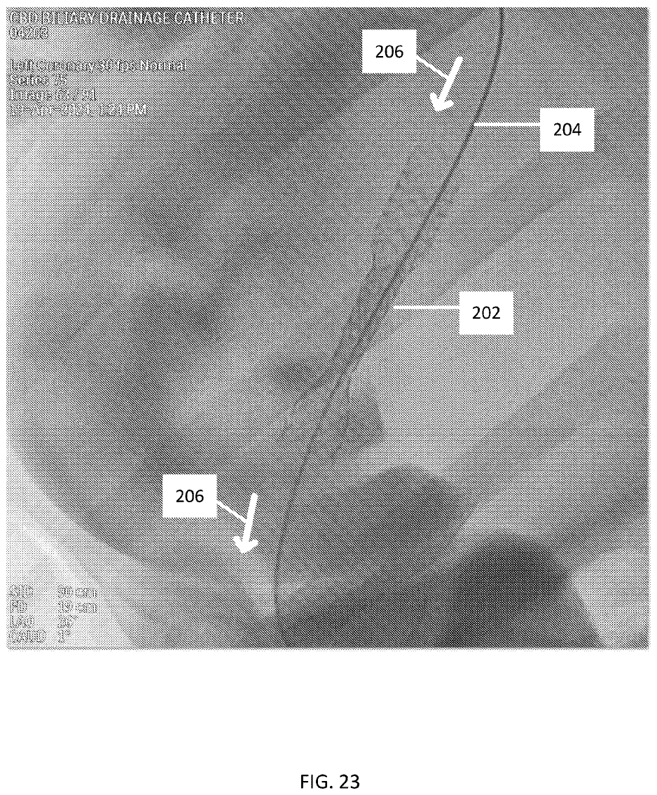

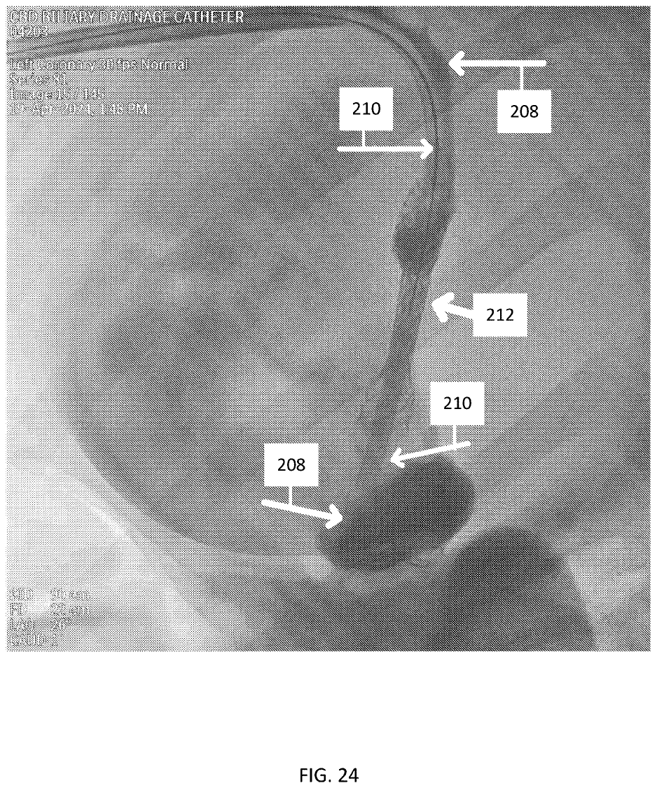

FIGS. 23 and 24 show an experiment that was conducted to demonstrate the effectiveness of the flow augmentation system. FIG. 23 is a fluoroscopic image from contrast cholangiography via a biliary catheter in an adult porcine model of iatrogenic biliary stenosis. The biliary stenosis in this experiment has been created via hourglass deployment of a covered stent 202. A guidewire 204 traverses the covered stent 202, extending from the intrahepatic biliary system to the gut in the direction of arrows 206.

FIG. 24 is a fluoroscopic image of the porcine model after installation of the flow augmentation system, showing that the system is effective to actively move bile mixed with contrast from the liver to the gut. The gray colored contrast within the biliary system and gut is labeled, for example, at arrows 208. Arrows 210 indicate the two ends of the implanted drain catheter, inserted via the liver bile ducts and transversing the stenotic portion of the covered stent, extending to the gut. Arrow 212 indicates the position of the screw drive portion (similar to the screw drive portions shown in FIGS. 8-22) within the drain catheter. Activation of the system results in bile mixed with contrast moving from the liver to the gut.

Additions, deletions, substitutions, and other modifications may be made to the systems and methods described above without departing from the scope or spirit of the present inventions, which are set out in the following claims.

1. A method of facilitating in vivo liquid flow in a drain passageway having an obstruction, the method comprising:
(a) implanting a drain catheter and an elongated rotatable drive in the drain passageway, wherein the drain catheter extends through the obstruction, wherein the elongated rotatable drive extends along a lumen of the drain catheter;
(b) rotating the elongated rotatable drive such that liquid in the drain passageway is drawn into the drain catheter upstream of the obstruction and exits the drain catheter downstream of the obstruction.

2. The method of claim 1, wherein the drain passageway comprises an in vivo bile duct or an in vivo ureter.

3. The method of claim 2, wherein the obstruction is a tumor.

4. The method of claim 2, wherein the obstruction is a stenosis of the drain passageway.

5. The method of claim 1, wherein the elongated rotatable drive is implanted into the drain catheter after implantation of the drain catheter.

6. The method of claim 5, wherein the drain catheter comprises at least one lateral opening extending from an exterior of the drain catheter to the lumen.

7. The method of claim 6, wherein, after implantation of the drain catheter, at least one opening is located upstream of the obstruction.

8. The method of claim 7, wherein, after implantation of the elongated rotatable drive, during rotation of the elongated rotatable drive, liquid in the drain passageway enters into the drain catheter through the at least one upstream opening and exits the drain catheter downstream of the obstruction.

9. The method of claim 8, wherein the elongated rotatable drive remains in the drain catheter as the liquid drains through the drain catheter.

10. The method of claim 9, wherein the liquid flows unidirectionally through the drain catheter during rotation of the elongated rotatable drive and exits both through a lateral openings downstream of the obstruction and through a distal end of the drain catheter.

11. The method of claim 7, wherein the elongated rotatable drive includes a helical screw drive portion.

12. The method of claim 11, wherein, after implantation of the elongated rotatable drive, the screw drive portion extends through the obstruction and past at least one upstream opening and at least one downstream opening of the drain catheter.

13. The method of claim 11, wherein, after implantation of the elongated rotatable drive, the screw drive portion is located at or upstream of the obstruction.

14. The method of claim 11, wherein, after implantation of the elongated rotatable drive, the screw drive portion is located proximate the at least one opening of the drain catheter upstream of the obstruction.

15. The method of claim 11, wherein the elongated rotatable drive is coaxially inserted in the drain catheter.

16. The method of claim 11, wherein, prior to implantation of the elongated rotatable drive, an end segment of the elongated rotatable drive is trimmed off to shorten the elongated rotatable drive.

17. The method of claim 11, further comprising connecting the elongated rotatable drive to a drive motor.

18. The method of claim 17, wherein the drive motor periodically or continuously rotates the elongated rotatable drive.

19. The method of claim 18, wherein the drive motor is energized by a battery or a spring.

20. The method of claim 11, wherein the elongated rotatable drive is resiliently bendable.

21. The method of claim 1, further comprising quantifying a volume of fluid that has flowed through the drain catheter using a flow sensor associated with at least either the drain catheter or the elongated rotatable drive.

22. The method of claim 1, further comprising monitoring performance of the drain catheter using a feedback sensor.

23. A system for facilitating in vivo liquid flow in a drain passageway having an obstruction, the system comprising:
(a) an elongated drain catheter comprising a lumen, an exterior, and a plurality of lateral openings extending from the exterior to the lumen;
(b) an elongated rotatable drive, the elongated rotatable drive comprising a screw drive portion configured for insertion into the lumen of the drain catheter such that the screw drive portion extends past at least one of the lateral openings;
(c) a drive motor configured to rotate the elongated rotatable drive.

24. The system of claim 23, wherein the elongated rotatable drive is trimmable.

25. The system of claim 23, wherein the screw drive portion is resiliently bendable.
